(19) United States
(12) Patent Application Publication
(10) Pub. No.: US 2006/0216312 A1
(43) Pub. Date: Sep. 28, 2006

(54) Mutants of replication competent vaccinia virus

(76) Inventor: Bertram Jacobs, Tempe, AZ (US)

Correspondence Address: NEEDLE & ROSENBERG, P.C., SUITE 1000, 999 PEACHTREE STREET, ATLANTA, GA, 30309-3915, US

(21) Appl. No.: 11/199,852

(22) Filed: Aug. 8, 2005

Related U.S. Application Data
(63) Continuation of application No. PCT/US04/03897, filed on Feb. 9, 2004.
(60) Provisional application No. 60/445,758, filed on Feb. 7, 2003.

(51) Int. Cl. A61K 39/275; C12N 7/01

(52) U.S. Cl. 424/232.1; 435/235.1

(57) ABSTRACT

The present invention relates to vaccines having an increased level of safety comprising recombinant vaccinia viruses. The invention also relates to methods for stimulating a protective immune response in an immunized host using the vaccines of the invention. The vaccines and recombinant vaccinia viruses of the invention comprise a first nucleic acid comprising an expression control sequence and a second nucleic acid comprising an exogenous nucleic acid encoding a conditional replication gene product, wherein the expression control sequence is operably linked to the exogenous nucleic acid. The exogenous nucleic acid may, by its expression or non-expression, confer upon the recombinant vaccinia virus either sensitivity or dependence upon an exogenous molecule (e.g. a drug) or a condition. Importantly, to allow the recombinant vaccinia viruses of the invention to replicate normally under permissive conditions, the exogenous nucleic acid is inserted into a non-essential locus, e.g., the E2L/E3L inter-genic locus, K1L/K2L inter-genic locus, the superoxide dismutate locus, and the 7.5 K locus.

### SPECIFICATION

This application claims priority to U.S. Provisional Patent application No. 60/445,758 filed Feb. 7, 2003.

### FIELD OF THE INVENTION

The present invention relates to the field of improved vaccines against smallpox, particularly vaccines comprising vaccinia virus mutants that may be more safely administered to immune-competent and immune-compromised subjects.

### BACKGROUND OF THE INVENTION

There is a possibility, hopefully remote, that smallpox, and other pathogens will be used as bioterrorist weapons (19). Vaccines offer the best protection against these threats. Presently, however, conventional smallpox vaccines may be lethal for rare healthy individuals, and for immunosuppressed vaccinees, or contacts (14). It is the aim of this proposal to develop smallpox vaccines which are safe and efficacious for use in all individuals, especially the immunosuppressed. If this technology proves effective then in the future it will be possible to utilize recombinant DNA technology to introduce genes coding for other potential bioterrorism agents to permit simultaneous immunization against multiple targets.

Vaccinia virus (VV), the agent used in the smallpox vaccine, is one of the most effective vaccines ever used, having eliminated smallpox globally at less than 10 cents/dose. No cold chain is needed, and protection lasts for at least 10 years, and probably much longer (14). Nonetheless, there is a great need to develop safer strains of VV that are still effective in protecting against smallpox. Smallpox vaccine can cause serious complications in vaccinated individuals (20). Serious central nervous system abnormalities (encephalomyelitis and encephalopathy) occur in up to 1/20,000-1/100,000 otherwise healthy vaccinated individuals, with a case-fatality rate of 25-50% (14). Progressive vaccinia can occur in immune compromised individuals, including individuals with leukemia, Hodgkin's disease, lymphoma, HIV-infection and organ transplant recipients. Due to the high prevalence of HIV infection in many parts of the world the risk of transmitting vaccinia to these subjects is considerable, and would have disastrous effects. Progressive vaccinia, when it occurs, is almost uniformly fatal, despite treatment with vaccinia-immune globulin. Vaccinated individuals who have eczema may develop eczema vaccinatum (approximately 1/100,000 vaccinees), which has about a 5% mortality rate. The current smallpox vaccine is contra-indicated in pregnant women and children under one year of age (14). Since the vaccine is a live virus vaccine, complications can occur in non-vaccinated individuals who come in contact with recent vaccinees. Overall reportable complications occur in 1/1,000-1/10,000 vaccinees, with at least one death per million vaccines likely, with the strains of VV currently available for use (14).

Development of a safer smallpox vaccine is a tremendous challenge since the correlates of protection against smallpox are unclear, and it is impossible to test for protection against smallpox in humans, the natural host for variola, the smallpox virus. Furthermore, the relevance of animal models for protection of humans against smallpox is unclear. Thus, strains that are as closely related to the currently accepted vaccine strain, Dryvax, that are as immunogenic as Dryvax, but that are safer than Dryvax would have great potential as candidate next generation vaccines.

There are several candidate second generation vaccines. MVA was developed by multiple passage in chick embryo fibroblasts (41). MVA has lost the ability to replicate in most mammalian cells. While MVA is extremely safe for use in humans, its efficacy is unclear. It is unlikely that MVA, which is non-replicating, will be useful by scarification, the normal route for immunization with VV (13). Again, since the correlates of immunity for protection against smallpox are unknown, it will be difficult to determine if MVA can provide a strong enough and broad enough immune response to protect against smallpox. This is especially an issue with MVA since numerous genes have been interrupted in selection for MVA (5), and it is unclear if the gene products encoded by these interrupted genes might be required for development of an immune response that is protective against variola.

LC16m8 is a variant of W-Lister strain that has a small plaque, temperature sensitive phenotype (42-44). This virus also displays reduced neurovirulence in mice. The small plaque phenotype has been mapped to a gene in the HinDIII D fragment. The temperature sensitive and decreased virulence phenotypes map to an as yet unknown mutation. Again, while this virus is likely safer than wtVV for use in humans its efficacy is unclear. Since the LC16 m8 strain does not produce extracellular enveloped virions, which may be necessary for induction of a protective immune response (39), the efficacy of this strain is also questionable (11).

Several mutations in the VV E3L gene have been prepared (37). These mutations decrease neurovirulence by 3 to over 5 logs, compared to wtVV (9). Several of these viruses are avirulent after intra-nasal infection of SCID mice (LD₅₀>10⁶ pfu, compared to an LD₅₀ of 10² pfu for wild type VV, unpublished observations). Despite their decreased virulence these viruses form pocks after vaccination of mice by scarification and protect against challenge with wtVV (unpublished observations). However, again, since the relevance of animal models to protection against smallpox in humans is unclear, the efficacy of virus strains containing these mutations is unclear.

An alternative strategy for development of safer, effective vaccines against smallpox is to engineer conditional mutants of VV. Under permissive conditions these viruses would be indistinguishable from wild type, and thus, should produce an equivalent immune response. Under restrictive conditions these viruses would not replicate and thus would be avirulent.

Numerous conditional mutants of VV have been described. Esteban has expressed the cellular anti-viral enzyme PKR from an IPTG-inducible promoter (22). In the absence of IPTG this virus is wild type in cells in culture, but induction with IPTG interrupts virus replication (IPTG-sensitive virus). Traktman has placed the A14 gene (a W gene that is essential for replication) under control of a tet-inducible promoter, in which the repressor falls off in the presence of tetracycline/doxycycline (45). This virus required tetracycline for replication in cells in culture (tet-dependent virus). Metzger et al. (25) have shown that expression of the human cytomegalovirus (HCMV) UL97 gene in W induces sensitivity to ganciclovir in cells in culture. This virus should be sensitive to the orally available drug, valganciclovir. In a similar manner expression of the herpes simplex virus-thymidine kinase gene (HSV TK) should induce sensitivity to the orally available drug, acyclovir. Acyclovir has the added advantage of causing very few side-effects compared to ganciclovir (and presumably valganciclovir). Condit has shown that virus containing mutations in G2R are dependent on the drug IBT for replication (6, 12). No animal studies have been reported for any of the conditional lethal mutant viruses described above. Finally, a strain of VV that is exquisitely sensitive to treatment with IFN has been prepared (wtVV is IFN-resistant) (unpublished observations). This virus induces a protective immune response in immunized mice.

### SUMMARY OF THE INVENTION

Replication competent vaccinia virus (VV), the current vaccine for smallpox, can cause severe complications after vaccination, especially in immune suppressed individuals (14). The present invention provides a means for inducing a protective immune response under permissive conditions while providing a means for rendering the virus incapable of replication under non-permissive conditions. The present invention provides conditional mutants of VV that may be either drug-dependent or drug-sensitive. For example, for drug-sensitive viruses, a drug to which the vaccine virus is sensitive may be administered to vaccinated individuals who experience complications. Alternatively, for drug-dependent viruses, vaccninated individuals would receive a maintenance dose of the drug on which the virus is dependent. The drug-dependent virus may be rendered incapable of continued replication in individuals who experience complications simply by withdrawing administration of the maintenance drug.

Drug-dependent viruses have the added advantage that they would not be able to spread in a viable form from vaccinated individuals to contacts. The relative immunogenicity and safety of these two strategies may be compared with that of a current vaccine (Dryvax) in immunocompetent mice (immunogenicity and safety), and in immunodeficient SCID mice (safety only). In some preferred embodiments of the invention, strains of the invention may be engineered into a virus background suitable for use in humans, prepared under good manufacturing protocol (GMP) conditions and tested in chimpanzees and humans for safety and immunogenicity, compared to Dryvax.

The present invention provides a recombinant vaccinia virus comprising:
a first recombinant nucleic acid comprising a first expression control sequence and a conditional replication nucleic acid encoding a conditional replication gene product wherein the first expression control sequence is operably linked to the conditional replication nucleic acid encoding a conditional replication gene product; and
a second recombinant nucleic acid comprising a second expression control sequence and a nucleic acid encoding a transcription factor wherein the second expression control sequence is operably linked to the nucleic acid encoding a transcription factor,
wherein the transcription factor conditionally binds to the first expression control sequence and the second recombinant nucleic acid is in (a) the E2L/E3L inter-genic locus, (b) the K1L/K2L inter-genic locus, (c) the superoxide dismutase locus, (d) the 7.5K locus, or (e) any other non-essential region of the vaccinia viral genome. The first expression control sequence may comprise a tet response element and the transcription factor is selected from the group consisting of a tet repressor and a reverse tet repressor. Alternatively, the first expression control sequence may comprise a lac operator and the transcription factor may be a lac repressor.

In addition, the first expression control sequence may be a viral early/late promoter and the conditional replication nucleic acid may be an A14 gene. The first expression control sequence may also be a viral late promoter in which case the conditional replication nucleic acid may be a suicide gene selected from the group consisting of a constitutively-active cellular anti-viral human protein kinase p68 gene, an RNase A, a DNase I, an interferon-inducible nitric oxide synthase (iNOS), an eIF2α (S51D), an anti-sense A14R gene, a constitutively active caspase 3, and interferon-γ. Where the first recombinant nucleic acid comprises a suicide or other exogenous gene, it may be independently in (a) the E2L/E3L inter-genic locus, (b) the K1L/K2L inter-genic locus, (c) the superoxide dismutase locus, (d) the 7.5K locus, or (e) any other non-essential region of the vaccinia viral genome. The invention additionally provides a vaccine against smallpox or vaccinia virus comprising this recombinant vaccinia virus.

The invention also provides a recombinant vaccinia virus comprising:
a first nucleic acid comprising an expression control sequence and an exogenous nucleic acid encoding a conditional replication gene product,
wherein the expression control sequence is operably linked to the exogenous nucleic acid and the first nucleic acid is in (a) the E2L/E3L inter-genic locus, (b) the K1L/K2L inter-genic locus, (c) the superoxide dismutase locus, (d) the 7.5K locus, or (e) any other non-essential region of the vaccinia viral genome. In some preferred embodiments, the expression control sequence is a constitutive promoter and the exogenous nucleic acid is selected from the group consisting of a UL97 gene and an acyclovir-sensitivity gene. The invention additionally provides a vaccine against smallpox or vaccinia virus comprising this recombinant vaccinia virus.

In some embodiments of the invention, expression of the conditional replication nucleic acid renders the virus either dependent on, or sensitive to, a particular material or a particular condition. The conditional replication nucleic acid encodes a conditional replication gene product. In some embodiments of the invention, the recombinant vaccinia virus may have a deletion in the E3L gene.

The invention also provides a vaccine against smallpox and/or vaccinia virus comprising the foregoing recombinant vaccinia virus. The invention further provides methods of eliciting a protective immune response to smallpox virus and/or vaccinia virus comprising administering to an individual a recombinant vaccinia virus of the invention.

### BRIEF DESCRIPTION OF THE DRAWINGS

The present invention is also illustrated, but without limitation, by the following figures:

FIG. 1: Survival of C57B16 mice following intranasal infection with vaccinia virus. Groups of 5 C57BL/6 mice were infected with different doses of wild type VV and the 5 mutant VV, by intranasal route. There was 100% survival of mice infected with the highest dose (10⁶) of the mutant viruses while wild type VV had an LD₅₀ of approximately 10³ pfu. The mutant VV constructs were over 1000 fold less pathogenic than wild type VV.

FIG. 2: Tissue distribution of virus. Groups of 3 C57BL/6 mice were infected with 10⁶ plaque forming units of wild type VV and 5 mutant VV constructs by the intranasal route. Tissues were harvested, processed and titrated in RK-13 cell line. The figure represents the average titer per gram of tissue of the 3 mice infected with each virus. Wild type VV was detected in the nasal turbinates, lungs and brain by 5 days post infection. The VV mutants were detected in the nasal turbinates but they did not spread to the lung and brain. 4 of the 5 VV mutants replicated to high titers in the nose following infection.

FIG. 3: Survival of mice following intracranial infection with the various recombinant VV. Groups of 5 C57 BL/6 mice were infected with different doses of wild type VV and 5 different mutants of VV, by intra cranial injection. The infected mice were observed for 2 weeks following infection and all mortalities were recorded. Mutants were from 3 logs to greater than 5 logs less neurovirulent than wtVV.

FIG. 4: Pathogenicity in SCID mice. Groups of SCID mice were infected intra-nasally (I.N.) with the indicated dose of virus and monitored for mortality for two weeks. Three of the viruses were apathogenic in SCID mice.

FIG. 5: Protection against challenge with wtVV. Groups of four week old C57B16 female mice were immunized I.N. with the indicated dose of virus or were mock immunized. Four weeks later animals were either mock challenged, or were challenged with 10⁶ pfu of VV-WR and monitored for ten days for weight loss.

FIG. 6: Interferon-sensitivity of VVΔE3L-ATV-1HD. Monolayers of RK-13 cells were pre-treated with the indicated concentration of IFNβ and then infected with approximately 100 pfu of the indicated viruses. Plaques were counted after 48 hours. wtVV is completely IFN-reisistant, while the mutant virus forms plaques in the absence of IFN, but not the presence of IFN.

FIG. 7: HCV C-NS3 specific cellular immune responses induced by different priming/boosting regimes of recombinant vaccinia and canarypox vectors.

FIG. 8: Immune response to vaccinia 2 weeks post challenge in chimp 157.

FIG. 9: HCV-specific responses in chimpanzees.

FIG. 10. Schematic representation of construction of pMPE3LEx-tetR.

### DETAILED DESCRIPTION OF THE INVENTION

The present invention provides vaccines against smallpox and vaccinia virus comprising a recombinant vaccinia virus. A “recombinant vaccinia virus” of the invetion comprises a first nucleic acid comprising an expression control sequence, and a second nucleic acid comprising an exogenous nucleic acid encoding a conditional replication gene product that renders the vaccinia virus either drug-sensitive or drug-dependent, wherein the expression control sequence is operably linked to the exogenous nucleic acid.

According to the invention “gene product” refers to the biochemical material(s) that result(s) from expression of a gene. It includes without limitation both nucleic acids (e.g. mRNA, rRNA, tRNA, and RNAi) and peptides (e.g. short peptides, polypeptides, and proteins). Protein gene products include without limitation preproproteins, proproteins, and mature proteins.

According to the invention, “conditional replication gene product” refers to a gene product upon which continued existence of the vaccinia virus in the mammalian host environment depends. This term also refers to a gene product to which continued existence of the vaccinia virus is sensitive. Viral existence may depend on, for example, replication of the viral genome, packaging of the viral genome, and expression of viral genes. In addition, viral existence may depend on exposure and/or susceptibility of viral nucleic acids to host nucleases. Viral existence may further depend on viability of the host cell. Exogenous genes encoding conditional replication gene products of the invention are located, not in the host genome, but in the viral genome. The exogenous gene may be, in its entirety, from one or more non-vaccinia virus sources. Alternatively, it may be a recombinant version of a gene native to vaccinia virus (e.g. A14).

In some embodiments of the invention, the recombinant vaccinia virus is dependent on expression of an exogenous gene (i.e. formation of the conditional replication gene product). In other embodiments, the recombinant vaccinia virus is sensitive to the expression of the exogenous gene such that virus and/or host cell are killed upon expression of the exogenous gene (e.g. a suicide gene).

In presently preferred embodiments of the invention, dependence on or sensitivity to the conditional replication gene product may depend on the presence of an exogenous factor. Thus, continued existence of the cell may depend on the presence of the conditional replication gene product and a drug, such that upon withdrawal of the drug, the conditional replication gene product is either no longer functional or no longer produced. Alternatively, continued existence of the cell may be sensitive to the presence of a drug in combination with the conditional replication gene product such that either one alone is harmless, but the combination terminates viral replication and/or kills the host cell.

Conditional replication gene products of the invention may affect viral existence positively or negatively. In the former case, the conditional replication gene product may support the continued existence of the virus so long as the requisite condition is met (e.g. adequate amount of a drug or nutrient). In the absence of the requisite condition, the gene product is not produced in a functional form or is rendered non functional such that viral replication is terminated.

In the latter case, the gene product terminates or facilitates termination of the virus. In preferred embodiments of the invention, the conditional existence gene is regulated by an expression control sequence such that its expression is inducible (e.g. expression is induced by the presence of a drug).

Expression control sequences of the invention include, without limitation, promoters, enhancers, transcription binding sites, and terminators. Expression control sequences of the invention may comprise vaccinia viral early/late promoters, viral late promoters, tet response elements, lac operators, and other inducible and constitutive promoters.

Nucleic acids encoding conditional replication gene products of the invention include, without limitation, essential viral genes, suicide genes, drug-sensitivity genes, and cytotoxic genes. A nonlimiting example of an essential viral gene is the A14 gene. Nonlimiting examples of viral suicide genes include a constitutively-active cellular anti-viral human protein kinase p68 (PKR) gene (56), an RNase A gene, a DNase I gene, an interferon-inducible nitric oxide synthase gene (iNOS), an eIF2α (S51D) gene (this is essentially a dominant negative inhibitor)(57), an anti-sense construct of an essential gene (such as A14R), a constitutively active caspase 3 gene (58), and an interferon-γ gene. Nonlimiting examples of drug-sensitivity genes include the UL97 gene of HCMV (ganciclovir/valganciclovir sensitivity) and the HSV acyclovir-sensitivity gene (HSV TK).

The present invention utilizes known inducible transcription systems including the tet repressor system and the lac repressor system. While variants of the tet repressor system may be used in the practice of the invention, the appended non-limiting examples refer to the classical system in which the repressor protein is bound to the tet response element in the absence of tetracycline, thereby repressing transcription of the subject gene. Conversely, when drug is present, the tet repressor protein binds to the drug, not the tet repressor element, thereby allowing removing the impediment to transcription. These inducible transcription systems of the invention may use transcription factors including transcription activators and transcription repressors. Nonlimiting examples of transcription repressors of the invention include the tet repressor, the reverese tet repressor, and the lac repressor. The mutated or “reverse” tetR gene binds to the Tet response element and suppresses transcription in the presence of doxycycline (27).

The lac repressor has been successfully used in mammals (59-61). As with the tet repressor system, the lac repressor system and its variants may be used in the practice of the instant invention.

In some embodiments of the invention, the recombinant vaccinia virus may lack a portion of the E3L gene. In particular, the invention provides a recombinant vaccinia virus in which a portion of the E3L gene is replaced with the eukaryotic initiation factor 2α gene (eIF2α) of Ambystoma tigrinum virus (ATV). These recombinant viruses may be interferon sensitive, but possess a broad host range, thus partially rescuing the phenotype of VV deleted for E3L gene. According to some nonlimiting examples of the invention, replacing the E3L gene of VV with the eIF2α homolog partially restored the wild type phenotype to the recombinant virus. The E3L gene of VV provides IFN resistance, a wide host range phenotype and inhibits apoptosis (Kibler et al., 1997, J Virol 71(3):1992-2003; Shors et al., 1997, Virology 239(2):269-76). It also functions as an inhibitor of PKR (Chang et al., 1992, Proc Natl Acad Sci USA 89(11):4825-9; Romano et al., 1998, Mol Cell Biol 18(12):7304-16), OAS (Rivas et al., 1998, Virology 243(2):406-14) and IRF-3 phosphorylation (Smith et al., 2001, J Biol Chem 276(12):8951-7). Thus, these recombinant viruses may resemble the wtVV in having a broad host range and in inhibiting PKR activity. At the same time these recombinant viruses may also resemble VVΔE3L in being IFN sensitive and leading to OAS activity and IRF-3 translocation to the nucleus. However, E3L-deleted viruses, though capable of replication, may not replicate to the same levels as wild-type virus.

Recombinant vaccinia viruses of the present invention may be constructed by methods known in the art, and preferably by homologous recombination. Standard homologous recombination techniques utilize transfection with DNA fragments or plasmids containing sequences homologous to viral DNA, and infection with wild-type or recombinant vaccinia virus, to achieve recombination in infected cells. Conventional marker rescue techniques may be used to identify recombinant vaccinia virus. Representative methods for production of recombinant vaccinia virus by homologous recombination are disclosed by Piccini et al., 1987, Methods in Enzymology 153:545.

Vaccinia virus used for preparing the recombinant vaccinia virus of the invention may be a naturally occurring or engineered strain. Strains useful as human and veterinary vaccines are particularly preferred and are well-known and commercially available. Such strains include Wyeth, Lister, WR, and engineered deletion mutants of Copenhagen such as those disclosed in U.S. Pat. No. 5,762,938.

Recombination plasmids may be made by standard methods known in the art. The nucleic acid sequences of the vaccinia virus known in the art, and may be found for example, in Earl et al., 1993, in Genetic Maps: locus maps of complex genomes, O'Brien, ed., Cold Spring Harbor Laboratory Press, 1.157 and Goebel et al., 1990, supra. The vaccinia virus used for recombination may contain other deletions, inactivations, or exogenous DNA.

Following infection and transfection, recombinants can be identified by selection for the presence or absence of markers on the vaccinia virus and plasmid. Recombinant vaccinia virus may be extracted from the host cells by standard methods, for example by rounds of freezing and thawing.

The present invention provides conditional mutants of VV and methods of preparing such conditional mutants. In some embodiments of the invention, these mutants may be tested for pathogenesis under permissive and restrictive conditions, and for their ability to induce a protective immune response against a wt-VV challenge, compared to Dryvax. Since under permissive conditions these viruses should be equivalent to a wtVV, they are expected to demonstrate equivalent efficacy and immunogenicity as compared to Dryvax. This is expected to make testing for efficacy more straightforward than for any other candidate vaccines. For drug dependent viruses, should complications arise, the drug could be removed, yielding viruses that no longer replicate. These drug dependent viruses also should be apathogenic if accidentally spread to susceptible individuals. For drug sensitive viruses, treatment with the FDA-approved drug (i.e., tetracycline/doxycycline, acyclovir, ganciclovir, valganciclovir or IFN) should decrease symptoms in individuals diagnosed with complications.

According to the invention, it may be possible to produce vaccinia virus strains that are sensitive to any molecule based on the expression or non-expression of an exogenous gene. In some preferred embodiments, the molecule selected is suitable for administration to humans and other subjects. The molecule may be of any size, structure, charge, and pI, and may be hydrophobic, hydrophilic, or amphipathic. The molecule may comprise amino acids, lipids, nucleic acids, sugars and other carbohydrates. In some preferred embodiments, the molecule is a drug. Nonlimiting examples of presently preferred drugs to which vaccinia virus strains may be rendered sensitive include doxycycline, acyclovir, ganciclovir/valganciclovir, interferon, and IPTG.

According to the invention, it may be possible to produce vaccinia virus strains that are dependent on any molecule. In some preferred embodiments, the molecule selected is suitable for administration to humans and other subjects. The molecule may be of any size, structure, charge, and pI, and may be hydrophobic, hydrophilic, or amphipathic. The molecule may comprise amino acids, lipids, nucleic acids, sugars and other carbohydrates. In some preferred embodiments, the molecule is a drug. Nonlimiting examples of presently preferred drugs to which vaccinia virus strains may be rendered dependent include doxycycline and IPTG.

According to the invention, a tetracycline analog includes without limitation tetracycline, doxycycline, and minocycline. The invention further contemplates the use of other modified forms of tetracycline that are capable of binding the either wild-type of modified forms of the tet repressor.

In some embodiments, the invention provides an assay for pathogenicity in immunocompetent and SCID mice or other mammals, and immunogenicity and protective efficacy of engineered viruses in an immunocompetent mammal under treated and untreated conditions.

The present invention provides methods for the preparation of a GMP batch of the vaccinia virus strains of the invention. According to some preferred embodiments of the invention, safety and immunogenicity may be tested in chimpanzees or in other primates or other mammals.

The present invention further provides vaccines for providing immunological protection against vaccinia virus or valiola virus, wherein said vaccines comprise a recombinant vaccinia viral vector and a carrier. The term carrier as used herein includes any and all solvents, diluents, dispersion media, antibacterial and antifungal agents, microcapsules, liposomes, cationic lipid carriers, isotonic and absorption delaying agents, and the like. Suitable carriers are known to those of skill in the art. The vaccine compositions of the invention can be prepared in liquid forms, lyophilized forms or aerosolized forms. Other optional components, e.g., stabilizers, buffers, preservatives, flavorings, excipients and the like, can be added. In addition, adjuvants may be used to boost or augment immune responses. Optionally, the vaccine may be formulated to contain other active ingredients and/or immunizing antigens.

Also included in the invention is a method of vaccinating a host, including but not limited to mammals such as humans, against vaccinia virus infection and/or variola virus infection with the novel vaccine compositions of the invention. The vaccine compositions, including one or more of the recombinant vaccinia viruses described herein, are administered using routes typically used for immunization, i.e., subcutaneous, oral, or nasal administration, in a suitable dose. The dosage regimen involved in the method for vaccination, including the timing, number and amounts of booster vaccines, may be determined considering various hosts and environmental factors, e.g., the age of the patients, time of administration and the geographical location and environment. In addition the present invention includes methods and compositions for stimulating in an individual an immune reponse.

The present invention contemplates phase I and phase II clinical trials in immunocompetent and immunosuppressed (e.g. HIV related) subjects with recombinant vaccinia virus strains of the invention.

### EXAMPLES

This invention will be better understood from the following examples. However, one skilled in the art will readily appreciate the specific materials and results described are merely illustrative of, and are not intended to, nor should be intended to, limit the invention as described more fully in the claims which follows thereafter.

### Example 1

### Engineering of vaccinia Virus Containing Mutations in the E3L Gene

Over 50 strains of VV containing mutations in the E3L gene have been prepared. Mutations are generally prepared by transient dominant selection, using the ecogpt gene as the transient selectable marker. Plasmid containing mutations in the E3L gene are used to perform homologous recombination with viruses containing an E3L gene replaced by lacZ, transiently selecting for resistance to mycophenolic acid (selection for ecogpt), and then screening for loss of staining with X-gal (replacement of lacZ by the gene of interest). Alternatively, virus that has incorporated a wild type E3L gene can be selected for by growth on Vero or MRC-5 cells (virus deleted for E3L does not replicate in Vero or MRC-5 cells). All viruses are assayed by PCR for the correct insertion, for the correct phenotype in cells in culture and by sequence analysis of the inserted gene.

### Example 2

### Safety

The present inventor has previously tested strains of VV for safety and efficacy in mouse model systems. Most of this work has been done with viruses containing mutations in the VV E3L gene and demonstrates an ability to engineer numerous strains of VV and to test them for safety, in both immune competent and immune deficient mice, and for efficacy as vaccines in a mouse challenge model.

Although a subset of the examples relate to viruses containing mutations in the E3L gene, the Examples are included to illustrate the materials and methods that may be used to generate and assay the activity of recombinant viruses of the invention.

Four mouse models have been used to assay for safety of engineered strains of VV: intra-nasal (I.N.) and intra-cranial (I.C.) infection of C57B16 mice, intra-nasal infection of Balb/c mice and intra-nasal infection of SCID mice. The LD50 of wtVV-WR in these models is as follows:

| Type of infection | LD₅₀ (pfu) |
|---|---|
| wtVV-WR/intra-nasal/C57B16 | 10⁴ |
| wtVV-WR/intra-cranial/C57B16 | 10-10² |
| wtVV-WR/intra-nasal/Balb/c | 10³ |
| wtVV-WR/intra-nasal/SCID | 10 |

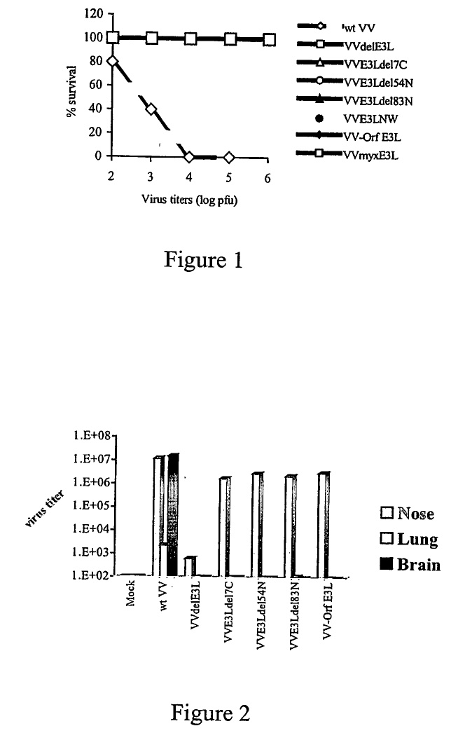

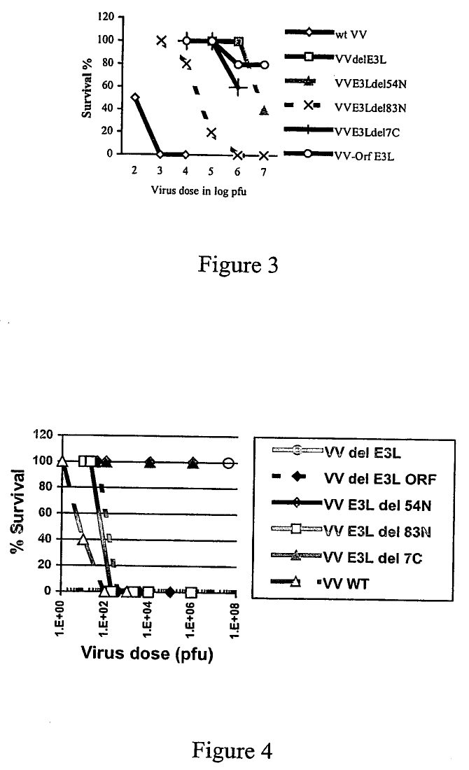

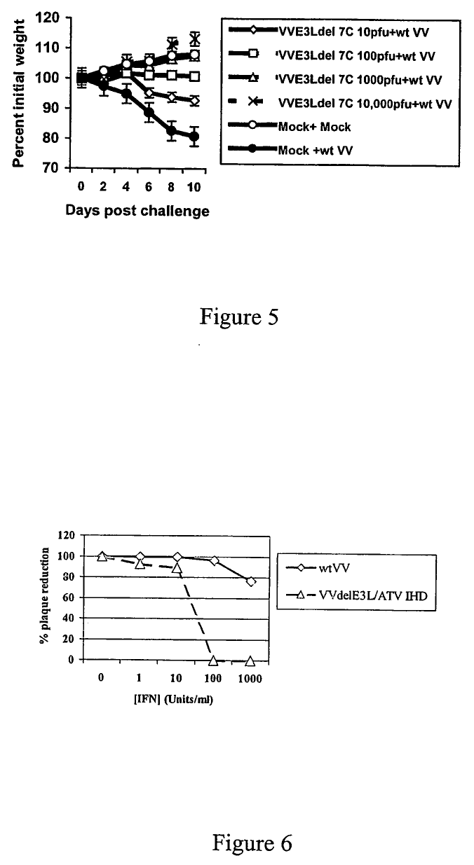

The I.N. models (FIGS. 1, 2) have the advantage over the I.C. models (FIG. 3) in that they require spread from the site of infection (FIG. 2) to get morbidity or mortality. Thus, they mimic natural disease or complications more closely than I.C. infection. Balb/c mice are less immune competent than C57B16 mice, and thus the assay is more sensitive in Balb/c mice. Infection of SCID mice allows analysis in a state of immune deficiency. I.C infection is much more sensitive than I.N. infection, such that viruses that are apathogenic by the I.N. route, show varying degrees of pathogenicity by the I.C. route. I.N. infection of SCID mice is amongst the most sensitive assays performed. Some mutant viruses that are highly attenuated in immune competent mice are still relatively pathogenic in SCID mice. This suggests that safety must be tested in several animal models.

### Example 3

### Vaccine Efficacy

The C57B16 and Balb/c challenge models have been used to test for efficacy of putative vaccines. The Balb/c model has the advantage that wtVV-VVR causes mortality after I.N. infection in 8 week old mice, while in the C57B16 model, mice get sick and loose weight but do not die. Groups of four week old mice are vaccinated either I.N. or by scarification, and monitored for symptoms of the vaccine (weight loss, morbidity; formation of skin lesions for scarification). After four weeks, mice are challenged I.N. with a large dose (10⁶ p fu) of wtVV-WR and monitored for weight loss, morbidity and death (Balb/c). Vaccination afforded a dose dependent protection against weight loss induced by the wtVV challenge and afforded protection against death in Balb/c mice (data not shown). Protection was obtained with a lower dose of virus I.N. (10³ pfu) than by scarification (10⁶ pfu, data not shown)

### Example 4

### Interferon-Sensitive Vaccinia Virus

VV is amongst the most IFN-resistant viruses known. VV is also a poor inducer of IFN. A variant of VV that is replication competent, but that both induces high concentrations of IFN and is highly sensitive to IFN has been engineered. This virus has the VV IFN-resistance gene replaced by a putative inhibitor of host defenses (IHD) from the salamander virus ATV. The ATV IHD acts to inhibit one arm of the mammalian IFN system, the PKR pathway, but does not inhibit the other arms of the IFN pathway and does not inhibit induction of IFN (data not shown). This virus replicates to nearly wild type titers in the absence of IFN (FIG. 6), but is exquisitely sensitive to treatment with IFN (FIG. 6). Supernatant from cells infected with this virus inhibited replication of VSV. This inhibitory activity could be inactivated with anti-serum to IFNβ (data not shown), indicating that this virus is a potent inducer of IFN. This virus induced an immune response that was protective against a wtVV challenge (data not shown).

### Example 5

### Advantage of Replicating vs Non-Replicating VV for Immunogenicity

The immunogenicity of replicating and non-replicating poxviruses when used as boosters after DNA based priming has been compared. As shown in Table 1, replicating VV provided approximately a 10 fold stronger cell mediated immune response when tested 4 weeks after boosting, and a 30 fold enhancement when tested 6 months after boosting. Thus replicating pox viruses are not only more immunogenic, but also produce better long term immunological memory.

TABLE 1
HCV Cap-NS3 specific IFNγ secreting cells in different DNA
prime/boost regimens

| | ISC/10⁶ spenocytes (direct - ex vivo) | |
|---|---|---|
| Immunization | 4 weeks | 24 weeks |
| DNA/DNA w/o boost | 410 +/− 8 | 0 +/− 0 |
| DNA/DNA/DNA | 785 +/− 12 | 61 +/− 11 |
| DNA/DNA + r-canarypox boost | 3946 +/− 853 | 2125 +/− 12 |
| DNA/DNA + r-VV boost | 41000 +/− 212 | 78000 +/− 212 |

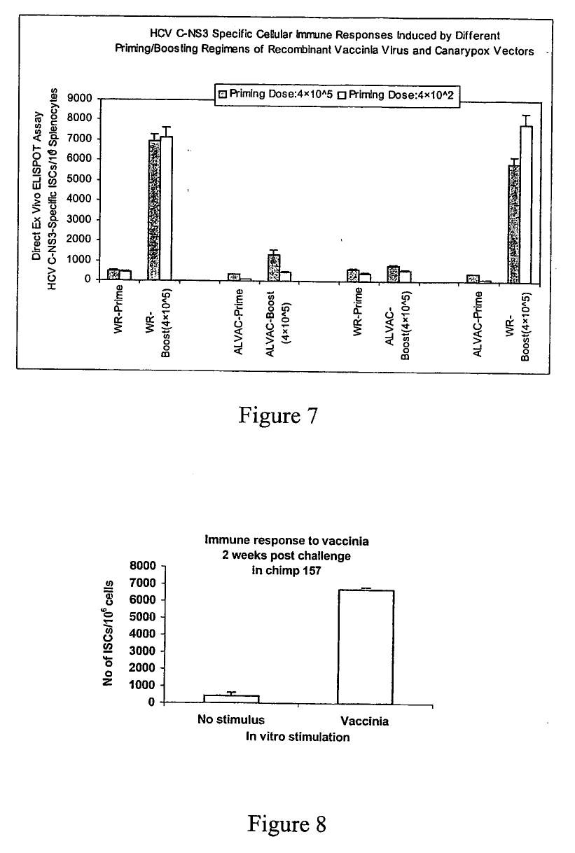

Also, the efficacy of priming with different doses of HCV recombinant canarypox or vaccinia followed by boosting with these viruses has been investigated. Canarypox prime-canarypox boost gave relatively weak responses, however canarypox priming followed by vaccinia boosting gave very strong responses, averaging 7-8000 IFN secreting cells/10⁶ PBMC. Similar levels of response were seen with vaccinia prime-vaccinia boost regimens. Optimal results were obtained with low dose (10² pfu) priming. The results are shown on FIG. 7.

### Example 6

### Cellular Immune Response to Vaccinia virus in Chimpanzee

The immune response of chimpanzee immunized with vaccinia vector 2 weeks post challenge was measured using IFN-γ ELISPOT assay. As shown in FIG. 8, the number of Vaccinia-specific interferon-γ secreting cells (ISCs) was dramatically increased 2 weeks post immunization.

### Example 7

### Prophylactic Immunization with Recombinant Vaccinia Viruses in Chimpanzees

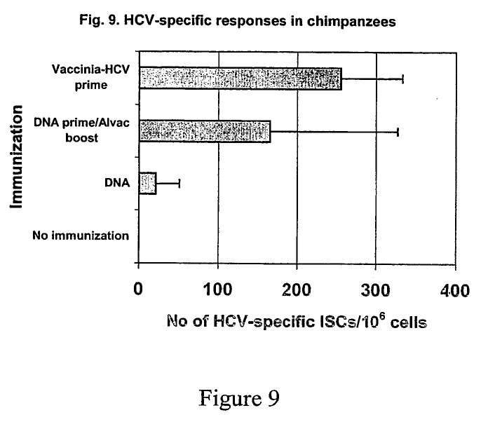

A trial of prophylactic immunization in 4 chimpanzees using a single dose of HCV, HBV, and HIV recombinant VVs has been initiated. Results obtained so far have revealed a superior cell mediated immune response to the transgene encoded antigen as shown in FIG. 9.

### Example 8

### Virus Construction

Several conditional mutants of VV may be prepared and tested in mouse models for safety, immunogenicity and efficacy. VV strains may then be engineered into a background appropriate for use in humans (Wyeth/NYCBOH/Acambis 2000), prepared under GMP conditions and tested for safety and immunogenicity in chimpanzees and humans. Four strains of VV may be prepared initially: a tet-dependent strain, a tet-sensitive strain, a ganciclovir/valganciclovir-sensitive strain and an acyclovir-sensitive strain. In addition, an IFN-sensitive virus may also undergo testing in mice. It should be noted that it may be possible to incorporate several of these safety features into a single virus, perhaps creating a vaccine strain that could be treated with either tetracycline, ganciclovir, acyclovir, or IFN, or any combination of these drugs. Furthermore, these strains may provide the basis for highly immunogenic but safe vectors for vaccinating against numerous agents, including other agents of bioterrorism.

Viruses may be initially prepared in a wtVV-WR background as VV-WR is the most appropriate strain to test for relative safety of mutant viruses. There are numerous model systems available for safety testing VV-WR mutants. The disadvantage of the WR strain is that it is not appropriate for use in humans. Thus, VV strains may be constructed in a Wyeth/NYCBOH/Acambis 2000 background.

### Example 9

### Virus Construction: Tetracycline-Dependent Virus

Traktman et al., described a tet-dependent VV that is TK-(45). However, since it is imperative for the constructs of the present invention to retain the ability to replicate normally under permissive conditions, the tet-repressor gene (tetR) is preferrably inserted into a non-essential locus of VV. This is not trivial since all of the insertion sites so far described for VV affect either virus replication or pathogenesis. Initially, tetR may be cloned downstream from the E3L ORF. tetR may also be cloned into a locus shown not to be necessary for pathogenesis in mice.

In addition, a tet-responsive element may be inserted between the transcription and translation start sites of the A14 gene of VV-WR, which has been shown to be essential to vaccinia virus morphogenesis (45).

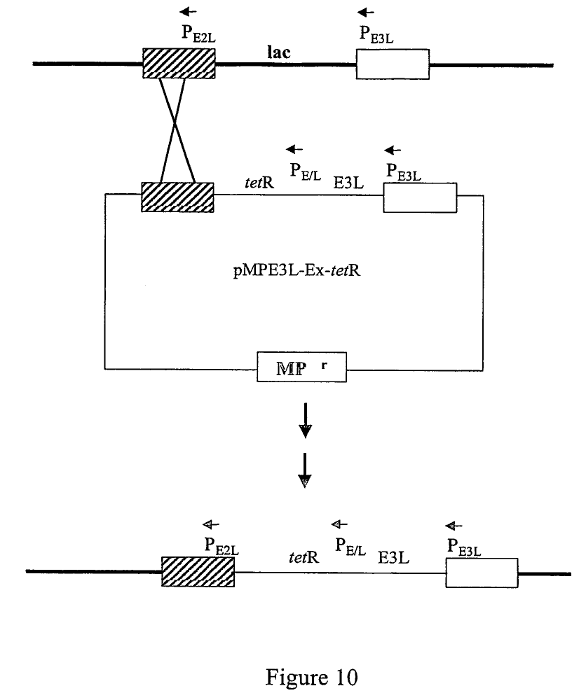

The tet-repressor may be inserted into an inter-genic site between the E2L and E3L genes. There are 140 bps of DNA between the end of the E3L ORF and the beginning of the E2L ORF. Since the E2L promoter is likely within 50 bps of the beginning of the E2L ORF, it is unlikely that insertion of genes immediately downstream of E3L will have any effect on either E3L or E2L gene expression. A cassette containing a synthetic VV early/late promoter, and the tetR gene followed by a VV transcription termination signal may be cloned into the unique restriction sites downstream of the E3L locus in the VV insertional plasmids pMPE3L to create pMPE3LEx-tetR (see FIG. 10). pMPE3L contains an E3L gene, flanked by unique cloning sites and by E3L right and left flanking arms for site specific recombination into the E3L locus of VV. The plasmid also contains an E. coli gpt gene for selection of viruses that have acquired the entire plasmid by a single homologous recombination event (ecogpt codes for resistance to mycophenolic acid). pMPE3LEx-tetR may be inserted into the E3L locus of VVΔE3L-lacZ (VV in which the E3L gene has been replaced by a lacZ gene) by homologous recombination. This may be accomplished by transfecting plasmid into CEF cells that have been infected with VVΔE3L-lacZ. Virus that has taken up plasmid by a single homologous recombination event may be selected by testing for resistance to mycophenolic acid and for replication in Vero cells (VVΔE3L does not replicate in Vero cells). Intra-molecular homologous recombination may be used to remove unwanted vector sequences and lacZ after removal of mycophenolic acid selection. Viruses that have resolved plasmid to replace lacZ with E3L and tetR may be identified by loss of staining with X-gal on Vero cells. Correct construction may be confirmed by PCR, Southern blot analysis and sequence analysis of the E3L locus. Expression of the E2L and E3L genes may be quantitated by Northern blot analysis. Dependence on tetracycline for replication in cells in culture may be assayed as previously described. Pathogenicity of this virus in the presence of doxycycline may be determined as described below.

The tetR gene may also be cloned into the SOD locus (A45R) of VV. The SOD locus has been shown not to be needed for replication in cells in culture or for pathogenesis in the mouse model (4). Thus cloning into the SOD locus is unlikely to have an effect on replication or spread in animals. A new insertional plasmid, which allows homologous recombination into the SOD locus in a manner similar to that described for the E3L locus, has been generated. Left and right SOD flanking arms have been inserted into the multiple cloning site of pBluescript. A cassette consisting of a VV synthetic early/late promoter, a gas gene (cleaves the chromogenic substrate X-glucuronic acid), a second synthetic early/late promoter and the tetR gene, may be inserted between the SOD left and right arms. After transfection/infection with this plasmid and wtVV, recombinants may be detected by staining with X-glucuronic acid. After three rounds of plaque purification of gas containing virus, virus that has resolved gus may be purified by selecting for clear plaques in the presence of X-glucuronic acid. Correct construction may be confirmed by PCR, Southern blot analysis and sequence analysis of the SOD locus. Phenotypic analysis may be performed as described above.

This tet-dependent system may be rendered tet-sensitive by simply substituting a reverse tetR gene for the tetR gene. The reverse tet repressor, as the name suggests, behaves a manner opposite from the tet repressor such that in the presence of tetracycline the repressor binds the TRE. Thus, in the absence of tetracycline or a suitable analog, the reverse repressor is not bound to the TRE thereby allowing A14 to be expressed. However, in the presence of tetracycline or a suitable analog, the reverese tet repressor binds TRE, which blocks expression of A14 and kills the virus.

### Example 10

### Virus Construction: Drug-Sensitive Viruses

The tet-dependent virus described in Example 9 should not be able to spread from vaccinated individuals to contacts and should complications arise, tetracycline can be withdrawn as a treatment. The disadvantage to a tet-dependent virus is that tetracycline must be given to all vaccinees. This could potentially be problematic for individuals with allergies to tetracycline. As an alternative several drug-sensitive viruses may be developed. Drug-sensitive viruses have the advantage that only patients who show signs of complications would be treated with the drug. Several different drug-sensitive viruses may be developed to determine which strategy yields the virus with the most desirable characteristics, i.e., wild type in the absence of drug, but with the best treatment profile. This may also have the advantage that in the future a multi-drug-sensitive virus could be developed, if necessary. It should be noted that an IFN-sensitive virus (WAE3L-ATV-1HD) has already been developed (55).

### Example 11

### Virus Construction: Drug-Sensitive Viruses: Tet-Sensitive Virus

Tet-sensitive W may be prepared in a similar manner as described for a tet-dependent virus, except that the tetR gene may be a reverse tet repressor, a mutated version that binds to the tet response element (TRE) and suppresses transcription in the presence of doxycycline (27).

A virus that expresses a suicide gene from a tet-regulated promoter may also be prepared. Esteban et al. have shown that expression of the cellular anti-viral protein PKR (interferon-induced human protein kinase p68) from a IPTG-inducible promoter in VV yields a virus that is isopropyl-β-D-thiogalactoside (IPTG) sensitive (IPTG induces expression of PKR which blocks virus replication) (22).

By contrast, the invention provides a virus in which expression of PKR under a tet-inducible promoter may yield a tet-sensitive virus. To prepare a virus with a tet-inducible PKR, the gene encoding PKR may be inserted into a non-essential locus under control of a TRE in a virus that contains the tetR (e.g. vaccinia virus constitutive promoter driving expression of tetR). Thus, in the absence of tetracycline or a suitable analog, the tet repressor binds to the TRE preventing expression of PKR. Accordingly, the virus is able to replicate in the host cell. However, upon exposure to tetracycline or a suitable analog, the tet repressor is not bound to the TRE allowing expression of PKR, which in turn kills the virus.

The tetR gene (tet-off) may be inserted in the E2L/E3L inter-genic locus. PKR may either be expressed from the 7.5K locus or the SOD locus. The SOD locus has not been found to be essential either in cells in culture or for pathogenesis in the mouse intra-nasal model. Thus, one would not expect insertion of PKR into the SOD locus to affect the ability of this virus to act as an immunogen. The 7.5K locus in VV-WR is a deleted version of the 35K locus present in Lister, but not WR, Wyeth or Tian Tian (28). Thus, the 7.5K locus in WR and Wyeth is likely not functional. Insertional vectors may be prepared with the PKR gene under control of a tet-inducible late promoter flanked by either SOD arms or 7.5K arms. Plasmids may contain gus as a transient dominant selectable marker, as described above. PKR may be inserted into virus by homologous recombination. Recombinant virus may be analyzed for replication in cells in culture in the presence and absence of doxycycline. Virus may be analyzed for pathogenesis in mice using the models described in a subsequent section, and may be analyzed for the ability to induce a protective immune response in mice.

This tet-sensitive system may be rendered tet-dependent by simply substituting a reverse tetR gene for the tetR gene. Thus, in the presence of tetracycline or a suitable analog, the reverese tet repressor binds TRE, blocking expression of PKR. In the absence of tetracycline or a suitable analog, the reverse repressor is not bound to the TRE thereby allowing PKR to be expressed.

### Example 12

### Virus Construction: Drug-Sensitive Viruses: Acyclovir, Ganciclovir/Valganciclovir Sensitive Viruses

Alternatively, the UL97 gene of HCMV (kindly provided by Adam Geballe, University of Washington) may be cloned into the site between E2L and E3L as described above. Virus may be characterized by PCR, Southern blot analysis and sequencing of the E2L/E3L loci, and by assaying for replication in cells in culture and pathogenicity and the ability to induce a protective immune response in mice in the absence of drug. Assays for sensitivity to ganciclovir in cells in culture may be performed as described (25). Sensitivity to ganciclovir and valganciclovir treatment in animals is described in Example 14.

Since ganciclovir and presumably valganciclovir both cause side-effects in some patients, a virus that expresses the HSV acyclovir-sensitivity gene (HSV TK) may also be prepared. The HSV TK gene may be cloned into the E2L/E3L inter-genic locus to prepare a virus potentially sensitive to acyclovir. While the literature has reported that the HSV TK gene has been inserted into VV, there do not seem to be references describing the acyclovir sensitivity of VV expressing the HSV TK. However, VV expressing the HSV TK can phosphorylate 5-iodo-2′deoxy-cytidine, a specific substrate for the HSV TK (29), and thus the HSV TK is likely active in VV. Virus may be assayed for replication in cells in culture and pathogenicity and the ability to induce a protective immune response in mice in the absence of drug. Virus may be assayed for sensitivity to acyclovir in a manner similar to assaying for ganciclovir sensitivity.

Both HSV and CMV are relatively GC rich (68% and 57% GC, respectively), while VV is AT rich (33% GC). If the high GC content of the CMV and HSV genes proves problematic (i.e., if virus expressing UL97 or TK is not wild type in cells in culture or in animals), AT rich versions of these genes may be prepared. Genes may be synthesized from overlapping oligonucleotides substituting most prominent codons utilized by VV for the UL97 or HSV TK codons. Synthetic genes may be cloned into the insertion site between E2L and E3L. Resulting virus may be screened for replication in cells in culture and pathogenicity and the ability to induce a protective immune response in mice in the absence of drug. Assay for sensitivity to ganciclovir or acyclovir in cells in culture may be performed as described (25).

### Example 13

### Animal Models for Testing for Testing WR Strain Conditional Mutants: Pathogenicity/Treatability

All constructs may be tested for pathogenicity, treatability, immunogenicity and induction of a protective immune response in mouse model systems.

Viruses may be tested for pathogenicity and treatability in at least four mouse model systems: I.N. and I.C. infection of 4-6 week old C57B16 mice; and intra-dermal (I.D.) and I.N. infection of SCID mice. The WR strain of VV is neurotropic in C57B16 mice. In mice infected I.N. with >104 pfu, virus spreads from the nose to the brain and animals die of encephalitis. This model may mimic post-vaccinial encephalitis in humans. The I.C. model in C57B16 mice may allow for testing of drug efficacy after the virus is in the CNS. This may be a model for treating patients who show signs of post-vaccinia encephalitis. The SCID mouse is a good model for testing pathogenicity and treatability in an immune compromised host.

For tet-dependent viruses, groups of C57B16 mice may be given doxycycline in their drinking water as previously described (33) (doxycycline is more stable than tetracycline), at −3, −2, −1 or 0 days pre-infection (animals may be infected I.N. with 10⁴-10⁶ pfu; app. 1-100 LD₅₀). Mice may be maintained on inducer through-out the course of the 14 day experiment. Animals may be monitored for weight loss, signs of morbidity, and compared to infection with wtVV-WR. Animals demonstrating greater than a 30% decrease in weight loss may be euthanized. A dose response experiment for treatment with inducer at day 0 may also be performed, using 0.1, 0.3, 1.0 and 3 mg/ml doxycycline. To test for the effect of removal of inducer, animals may be treated with the optimal concentration of inducer, infected and then inducer may be removed on day +1, +2, +4, +6 and +8.

The kinetics of spread of virus from the nose may be determined and compared to wtVV-WR. Groups of animals may be treated with the optimal regimen of inducer and infected I.N. with virus. Pairs of animals may be sacrificed every other day through 8 days, and nasal turbinates, lung, spleen, liver, heart, stomach, intestine, ovaries/testes and brain may be harvested. Virus may be released from tissue and titer on Vero cells in the presence of tetracycline. Spread may also be monitored by PCR for viral DNA.

Once the optimal regimen is determined in C57B16 mice, SCID mice may be infected I.N. or I.D. with 100 LD₅₀ of virus in the presence of inducer. Inducer may either be maintained through-out the course of the experiment or removed on days +2, +4, +6 or +8. Animals may be monitored as described above.

For tet-sensitive virus, groups of 4-6 week-old C57B16 mice may be infected with tet-sensitive or wtVV-WR. Animals may be infected I.N. with 10⁴-10⁶ pfu or I.C. with 10-10³ pfu (1-100 expected LD₅₀ in each case). This range of doses may permit determination of whether this virus is wild type for pathogenesis in the absence of treatment. At +2, +4, +6 or +8 days post-infection animals may be treated with the optimal dose of doxycycline, orally in their drinking water, or left untreated. Animals may be monitored for disease as described above. Among other things this may allow for assay of the efficiency with which encephalitis could be treated (both doxycycline and tetracycline pass the blood-brain barrier to some extent). Groups of SCID mice may be infected I.N. or I.D. with 1-100 LD₅₀ of virus (the I.N. LD₅₀ is expected to be 10 pfu in SCID mice; the I.D. LD₅₀ may be determined in preliminary experiments). Animals may be treated with doxycycline as described above, and monitored for weight loss and morbidity.

Similar experiments as described for tet-sensitive viruses may be performed for acyclovir, ganciclovir/valganciclovir-sensitive and IFN-sensitive viruses.

### Example 14

### Animal Models for Testing of WR Strain Conditional Mutants: Efficacy in a Mouse Challenge Model

Viruses may be tested for efficacy in the Balb/c I.D. vaccination, I.N. and I.P. challenge models. The Balb/c I.N. challenge model is the only VV model for which challenge is lethal in 8 week-old mice (older mice are necessary for vaccination/challenge models because vaccination is performed at 4 weeks and challenge is performed 4 weeks later). The I.P. model is very sensitive, in that small amounts of virus spreading to the ovaries may replicate to high tiers. Animals may be vaccinated by scarification with 10⁵-10⁷ pfu of virus (wtVV-WR, tet-dependent VV-WR, tet-sensitive VV-WR, acyclovir-sensitive VV-WR, ganciclovir/valganciclovir-sensitive VV-WR, VV-WRΔE3L-ATV-1HD (IFN-sensitive, see Preliminary Studies); 10⁶ pfu of wtVV-WR is required to protect mice from a wtVV challenge). For tet-dependent virus animals may be treated with the optimal regimen of inducer in their drinking water for two weeks. Animals may be monitored for weight loss and severity of pock at the site of infection (the base of the tail). Animals may be challenged I.N. at day +30 with 10⁵-10⁷ pfu of wtVV-WR (the I.N. LD₅₀ for 8 week-old mice is <10⁵ pfu). Animals may be monitored for 14 days for weight loss, morbidity and death.

For I.P. challenge, immunized and unimmunized control female mice may be challenged with 10⁷ pfu VV-VVR. After 5 days, their ovaries (where vaccinia replicates) are harvested, homogenized, and prepared for assay. Viral dilutions may be made ranging from 10⁻¹ through 10⁻¹⁰ and added to 5×10⁵ BSC/40 cells in 6 well plates for 4 hours at 37° C. 10% MEM (2.0 ml) is added back to wells and left at 37° C. for 3 days. Wells are then stained with crystal violet and plaques scored.

14.1. Evaluation of Cellular Immune Response, and Dose/Schedule Requirements, in Mice.

BALB/C mice may be inoculated with recombinant VV as described above. 3 mice may be sacrificed from each immunized group at 1, 2, 4 and 24 weeks post-vaccination. Spleens may harvested and plasma and splenocytes may be cryopreserved. Assays for cell mediated and humoral immunity may be carried out as described below for the chimpanzee study. A similar evaluation of immunogenicity may be done on mice receiving different doses of tetracycline to limit replication for different durations.

14.2. Evaluation of Immune Responses.

Drug requiring and drug sensitive vectors, and Dryvax as a control, may be evaluated for immunogenicity under conditions permitting viral replication for varying periods of time e.g. 14 days. For each of the vectors, the minimal replication time which produces optimal humoral and cellular immune responses may be determined. This may have the dual advantage of limiting the rate of complications in immunized individuals and minimizing spread to contacts. Groups of 3 mice immunized with different vectors under different drug regimens may be sacrificed 1, 2, 4, and 24 weeks after immunization. Spleens and plasma may be collected and splenocytes may be cryopreserved. Plasma may be assayed for anti-VV antibodies by Elisa, and by VV neutralization assays. Splenocytes may be evaluated for blastogenesis with VV proteins, interferon γ Elispot assays and CTL assays using VV sensitized target cells, intracellular cytokine staining, and cytokine secretion profiles. In addition immunized mice may be evaluated in a mouse protection assay by inoculation of wild type VV i.p. followed by quantitation of virus in ovaries by plaque assay. Selected samples may also be evaluated for T cell avidity, as this has been shown to be a major determinant of protective efficacy. Experiments utilizing the HLA 2.01 dependant VV epitope peptide (VP31#1, see below) for avidity determination may be carried out in A2 kb/H-2b HLA 2.01 transgenic mice.

### Example 15

### Animal Models for Testing of WR Strain Conditional Mutants: Reversion

Reversion of viruses to drug-independence or drug-resistance is a common problem, even with markers that provide a selective advantage to the virus. Thus, conditional mutants should be evaluated to determine if reversion is likely to be a problem. Tet-dependent and drug-sensitive viruses may be passaged in SCID mice under permissive conditions. Virus may be harvested from lung, brain, spleen and ovaries at various times post-infection. Virus may be titered in Vero cells under permissive and restrictive conditions to determine the fraction of revertant viruses that have evolved during passage in SCID mice. If there is frequent, large scale breakthrough of drug-independent or drug-resistant virus, the VV strain may be engineered further to be conditional for multiple treatments (i.e., tet-dependent, acyclovir-, valganciclovir-, IFN-sensitive) to minimize breakthrough of wild type virus.

### Example 16

### Engineer the Strain(s) into a Background Suitable for use in Humans:Preparation of Wyeth/NYCBOH Strains

Recombinant VV constructs demonstrating safety and efficacy may be prepared in a Wyeth/NYCBOH background, using the techniques described above for preparation of strains in a WR background.

Mutations may be made in a background virus selected from the group consisting of a Wyeth/NYCBOH virus stock, a reconstituted Dryvax stock, a Dynport vaccine, and Acambis 2000.

All stocks of Wyeth/NYCBOH may be maintained as a quasi-species. Wyeth/NYCBOH is a mixture of viruses with different phenotypes, and it is presently unclear which of the individual viruses would provide the optimal phenotype for a vaccine. Thus, as complex a mixture of viruses as possible will always be maintained. MOIs of at least 0.01 pfu/cell (greater than 10⁴ pfu/plate) may be used for growth of all stocks. For insertion of genes, entire plates of recombinant virus grown under selective conditions may be used for isolation of recombinants. This may ensure that hundreds of plaque variants are represented in the final recombinant virus. Recombinant constructs may be compared to the parent stock of Wyeth/NYCBOH by plaque morphology in cells in culture, and by restriction mapping to ensure the recombinant is composed of a quasi-species similar to the parental stock.

The sequence of the region surrounding E3L may be determined for the Wyeth/NYCBOH quasi-species that will be used for virus construction. If there are any differences between Wyeth and Copenhagen (the Copenhagen sequence was used to prepare recombination arms for insertion of genes into the region between E2L and E3L) then the corresponding changes will be made in pMPE3LEx to prepare a plasmid that will not introduce unwanted nucleotide changes into the Wyeth/NYCBOH background. The E2L and E3L loci of all recombinant quasi-species may be sequenced and compared to the parental stock.

All work with Wyeth/NYCBOH virus may be in either Vero cells or MRC-5 cells freshly obtained from ATCC using medium containing fetal calf serum from known, certified U.S. donor herds (kindly supplied by Aventis Pasteur). Work may be performed in a BSL-2 facility used only for development of vaccine strains, under strict GLP conditions.

### Example 17

### Manufacture of Recombinant Vaccinia Virus Constructs

In principle, the “Australian Code of GMP for Therapeutic Goods-Part II Sterile Products” is utilized as a main reference point for preparation of recombinant vaccinia virus for use in mammals and humans. However, there are aspects of GMP that are acknowledged as being different for Investigational Medicinal Products. Therefore, Appendix G “Guidelines for GMP for Investigational Medicinal Products of the Australian cGMP” is also utilized. The text of this document is that the Annexure “Manufacture of the Investigational Medicinal Products” to the Guide to GMP of the Commission of the European Communities DG 111/C/3.

### DOCUMENTS CITED

All sequences, patents, patent applications or other published documents cited anywhere in this specification are herein incorporated in their entirety by reference to the same extent as if each individual sequence, publication, patent, patent application or other published document was specifically and individually indicated to be incorporated by reference.
1. Alexander-Miller, M. A. 2000. Differential expansion and survival of high and low avidity cytotoxic T cell populations during the immune response to a viral infection. Cell Immunol 201:58-62.
2. Alexander-Miller, M. A., G. R. Leggatt, and J. A. Berzofsky. 1996. Selective expansion of high- or low-avidity cytotoxic T lymphocytes and efficacy for adoptive immunotherapy. Proc Natl Acad Sci USA 93:4102-7.
3. Alexander-Miller, M. A., G. R. Leggatt, A. Sarin, and J. A. Berzofsky. 1996. Role of antigen, CD8, and cytotoxic T lymphocyte (CTL) avidity in high dose antigen induction of apoptosis of effector CTL. J Exp Med 184:485-92.
4. Almazan, F., D.C. Tscharke, and G. L. Smith. 2001. The vaccinia virus superoxide dismutase-like protein (A45R) is a virion component that is nonessential for virus replication. J Virol 75:7018-29.
5. Antoine, G., F. Scheiflinger, F. Domer, and F. G. Falkner. 1998. The complete genomic sequence of the modified vaccinia Ankara strain: comparison with other orthopoxviruses. Virology 244:365-96.
6. Black, E. P., and R. C. Condit. 1996. Phenotypic characterization of mutants in vaccinia virus gene G2R, a putative transcription elongation factor. J Virol 70:47-54.
7. Blasco, R., and B. Moss. 1992. Role of cell-associated enveloped vaccinia virus in cell-to-cell spread. J Virol 66:4170-9.
8. Blasco, R., J. R. Sisler, and B. Moss. 1993. Dissociation of progeny vaccinia virus from the cell membrane is regulated by a viral envelope glycoprotein: effect of a point mutation in the lectin homology domain of the A34R gene. J Virol 67:3319-25.
9. Brandt, T. A., and B. L. Jacobs. 2001. Both carboxy- and amino-terminal domains of the vaccinia virus interferon resistance gene, E3L, are required for pathogenesis in a mouse model. J Virol 75:850-6.
10. Cohard, M., Q. Liu, M. Perkus, E. Gordon, B. Brotman, and A. M. Prince. 1998. Hepatitis C virus-specific CTL responses in PBMC from chimpanzees with chronic hepatitis C: determination of CTL and CTL precursor frequencies using a recombinant canarypox virus (ALVAC). J Immunol Methods 214:121-9.
11. Cohen, J. 2002. PUBLIC HEALTH: Looking for Vaccines That Pack a Wallop Without the Side Effects. Science 298:2314.
12. Condit, R. C., Y. Xiang, and J. 1. Lewis. 1996. Mutation of vaccinia virus gene G2R causes suppression of gene A18R ts mutants: implications for control of transcription. Virology 220:10-9.
13. Drexler, I., C. Staib, W. Kastenmuller, S. Stevanovic, B. Schmidt, F. A. Lemonnier, H. G. Rammensee, D. H. Busch, H. Bernhard, V. Erfle, and G. Sutter. 2003. Identification of vaccinia virus epitope-specific HLA-A*0201-restricted T cells and comparative analysis of smallpox vaccines. Proc Natl Acad Sci USA 100:217-22.
14. Fenner, F., D. A. Henderson, I. Arita, Z. Jezek, and I. D. Ladnyi. 1988. Smallpox and its eradication. World Health Organization, Geneva.
15. Frey, S. E., R. B. Couch, C. O. Tacket, J. J. Treanor, M. Wolff, F. K. Newman, R. L. Atmar, R. Edelman, C. M. Nolan, and R. B. Belshe. 2002. Clinical responses to undiluted and diluted smallpox vaccine. N Engl J Med 346:1265-74.
16. Frey, S. E., F. K. Newman, J. Cruz, W. B. Shelton, J. M. Tennant, T. Polach, A. L. Rothman, J. S. Kennedy, M. Wolff, R. B. Belshe, and F. A. Ennis. 2002. Dose-related effects of smallpox vaccine. N Engl J Med 346:1275-80.
17. Gray, P. M., G. D. Parks, and M. A. Alexander-Miller. 2003. High avidity CD8(+) T cells are the initial population elicited following viral infection of the respiratory tract. J Immunol 170:174-81.
18. Gray, P. M., G. D. Parks, and M. A. Alexander-Miller. 2001. A novel CD8-independent high-avidity cytotoxic T-lymphocyte response directed against an epitope in the phosphoprotein of the paramyxovirus simian virus 5. J Virol 75:10065-72.
19. Henderson, D. A. 1999. The looming threat of bioterrorism. Science 283:1279-82.
20. Lane, J. M., F. L. Ruben, J. M. Neff, and J. D. Millar. 1970. Complications of smallpox vaccination, 1968: results of ten statewide surveys. J Infect Dis 122:303-9.
21. Lee, D. H., and A. M. Prince. 2001. Automation of nucleic acid extraction for NAT screening of individual blood units. Transfusion 41:483-7.
22. Lee, S. B., R. Bablanian, and M. Esteban. 1996. Regulated expression of the interferon-induced protein kinase p68 (PKR) by vaccinia virus recombinants inhibits the replication of vesicular stomatitis virus but not that of poliovirus. J Interferon Cytokine Res 16:1073-8.
23. Lubaki, M. N., M. A. Egan, R. F. Siliciano, K. J. Weinhold, and R. C. Bollinger. 1994. A novel method for detection and ex vivo expansion of HIV type 1-specific cytolytic T lymphocytes. AIDS Res Hum Retroviruses 10:1427-31.
24. Lyons, A. B. 2000. Analysing cell division in vivo and in vitro using flow cytometric measurement of CFSE dye dilution. J Immunol Methods 243:147-54.
25. Metzger, C., D. Michel, K. Schneider, A. Luske, H. J. Schlicht, and T. Mertens. 1994. Human cytomegalovirus UL97 kinase confers ganciclovir susceptibility to recombinant vaccinia virus. J Virol 68:8423-7.
26. Neitzel, H. 1986. A routine method for the establishment of permanent growing lymphoblastoid cell lines. Hum Genet 73:320-6.
27. Paillard, F. 1998. “Tet-on”: a gene switch for the exogenous regulation of transgene expression. Hum Gene Ther 9:983-5.
28. Patel, A. H., D. F. Gaffney, J. H. Subak-Sharpe, and N. D. Stow. 1990. DNA sequence of the gene encoding a major secreted protein of vaccinia virus, strain Lister. J Gen Virol 71 (Pt 9):2013-21.
29. Perkus, M. E., D. Panicali, S. Mercer, and E. Paoletti. 1986. Insertion and deletion mutants of vaccinia virus. Virology 152:285-97.
30. Ramirez, J. C., M. M. Gherardi, and M. Esteban. 2000. Biology of attenuated modified vaccinia virus Ankara recombinant vector in mice: virus fate and activation of B- and T-cell immune responses in comparison with the Western Reserve strain and advantages as a vaccine. J Virol 74:923-33.
31. Reichstetter, S., W. W. Kwok, S. Kochik, D. M. Koelle, J. S. Beaty, and G. T. Nepom. 1999. MHC-peptide ligand interactions establish a functional threshold for antigen-specific T cell recognition. Hum immunol 60:608-18.
32. Rodriguez, J. R., C. Risco, J. L. Carrascosa, M. Esteban, and D. Rodriguez. 1997. Characterization of early stages in vaccinia virus membrane biogenesis: implications of the 21-kilodalton protein and a newly identified 15-kilodalton envelope protein. J Virol 71:1821-33.
33. Saitoh, Y., Y. Eguchi, Y. Hagihara, N. Arita, M. Watahiki, Y. Tsujimoto, and T. Hayakawa. 1998. Dose-dependent doxycycline-mediated adrenocorticotropic hormone secretion from encapsulated Tet-on proopiomelanocortin Neuro2A cells in the subarachnoid space. Hum Gene Ther 9:997-1002.
34. Sarkar, S., V. Kalia, M. Murphey-Corb, and R. C. Montelaro. 2002. Detailed analysis of CD4+ Th responses to envelope and Gag proteins of simian immunodeficiency virus reveals an exclusion of broadly reactive Th epitopes from the glycosylated regions of envelope. J Immunol 168:4001-11.
35. Sedlik, C., G. Dadagio, M. F. Saron, E. Deriaud, M. Rojas, S. I. Casal, and C. Leclerc. 2000. In vivo induction of a high-avidity, high-frequency cytotoxic T-lymphocyte response is associated with antiviral protective immunity. J Virol 74:5769-75.
36. Shata, M. T., D. D. Anthony, N. L. Carlson, L. Andrus, B. Brotman, N. Tricoche, P. McCormack, and A. Prince. 2002. Characterization of the immune response against hepatitis C infection in recovered, and chronically infected chimpanzees. J Viral Hepat 9:400-10.
37. Shors, T., K. V. Kibler, K. B. Perkins, R. Seidler-Wulff, M. P. Banaszak, and B. L. Jacobs. 1997. Complementation of vaccinia virus deleted of the E3L gene by mutants of E3L. Virology 239:269-76.
38. Slifka, M. K., and J. L. Whitton. 2001. Functional avidity maturation of CD8(+) T cells without selection of higher affinity TCR. Nat Inimunol 2:711-7.
39. Smith, G. L., A. Vanderplasschen, and M. Law. 2002. The formation and function of extracellular enveloped vaccinia virus. J Gen Virol 83:2915-31.
40. Speiser, D. E., D. Kyburz, U. Stubi, H. Hengartner, and R. M. Zinkernagel. 1992. Discrepancy between in vitro measurable and in vivo virus neutralizing cytotoxic T cell reactivities. Low T cell receptor specificity and avidity sufficient for in vitro proliferation or cytotoxicity to peptide-coated target cells but not for in vivo protection. J Immunol 149:972-80.
41. Stickl, H., V. Hochstein-Mintzel, A. Mayr, H. C. Huber, H. Schafer, and A. Holzner. 1974. [MVA vaccination against smallpox: clinical tests with an attenuated live vaccinia virus strain (MVA) (author's transl)]. Dtsch Med Wochenschr 99:2386-92.
42. Sugimoto, M., A. Yasuda, K. Miki, M. Morita, K. Suzuki, N. Uchida, and S. Hashizume. 1985. Gene structures of low-neurovirulent vaccinia virus LC16m0, LC16 m8, and their Lister original (LO) strains. Microbiol Immunol 29:421-8.
43. Takahashi-Nishimaki, F., S. Funahashi, K. Miki, S. Hashizume, and M. Sugimoto. 1991. Regulation of plaque size and host range by a vaccinia virus gene related to complement system proteins. Virology 181:158-64.
44. Takahashi-Nishimaki, F., K. Suzuki, M. Morita, T. Maruyama, K. Miki, S. Hashizume, and M. Sugimoto. 1987. Genetic analysis of vaccinia virus Lister strain and its attenuated mutant LC16 m8: production of intermediate variants by homologous recombination. J Gen Virol 68 (Pt 10):2705-10.
45. Traktinan, P., K. Liu, J. DeMasi, R. Rollins, S. Jesty, and B. Unger. 2000. Elucidating the essential role of the A14 phosphoprotein in vaccinia virus morphogenesis: construction and characterization of a tetracycline-inducible recombinant. J Virol 74:3682-95.
46. Wells, A. D., H. Gudmundsdottir, and L. A. Turka. 1997. Following the fate of individual T cells throughout activation and clonal expansion. Signals from T cell receptor and CD28 differentially regulate the induction and duration of a proliferative response. J Clin Invest 100:3173-83.
47. Jacobs B, “Methods of Inducing Apoptosis in a Target Cell” International Patent Publication No. WO 99/55910, published Nov. 4, 1999.
48. Jacobs B et al., “Recombinant Vaccinia Viral Vectors” International Patent publication No. WO 02/086059, published Oct. 31, 2002.
49. Jacobs B et al., “Recombinant Vaccinia Viral Vectors” Canadian Patent No. 2,444,945, published Oct. 31, 2002.
50. Rich A et al., “Method Of Inhibiting Pathogenicity Of Infectious Agents” U.S. Patent Publication No. 2003/211964, published Nov. 13, 2003.
51. Vyaysri S et al., “Viral Vectors Having Reduced Virulence” International Patent publication No. WO 02/085416, published Oct. 31, 2002.
52. Gettig R R et al., “Vectors Having Enhanced Expression And Methods Of Making And Uses Thereof” Australian Patent No. 745836, published Apr. 11, 2002.
53. Brandt T et al., “Novel Viral Vectors Having Enhanced Effectiveness With Dramatically Reduced Virulence” International Patent Publication No. WO 00/73487, published Dec. 7, 2000.
54. Brandt T et al., “Viral Vectors Having Enhanced Effectiveness With Reduced Virulence” U.S. Patent Publication No. 2002/0110565, published Aug. 15, 2002.
55. Jacobs B et al., “Use of Vaccinia Virus Deleted for the E3L Gene as a Vaccine Vector” International Patent Application No. PCT/US03/21784, filed Jul. 11, 2003.
56. Vattem, K. M., Staschke, K. A., Wek, R. C. (2001) Mechanism of activation of the double-stranded RNA dependent protein kinase, PKR. Role of dimerization and cellular localization in the stimulation of PKR phosphorylation of eukaryotic initiation factor-2 (eIF2). Eur. J. Biochem. 268:3674-3684.
57. Srivastava, S. P., Kumar, K. U. and Kaufian, R. J. Phosphorylation of eukaryotic initiation factor 2 mediates apoptosis in response to activation of the double-stranded RNA-dependent protein kinase. J. Biol. Chem. 273:2416-2423. 58. Srinivasula S M, Ahmad M, MacFarlane M, Luo Z, Huang Z, Fernandes-Alnemri T, Alnemri ES. Generation of constitutively active recombinant caspases-3 and -6 by rearrangement of their subunits. J Biol. Chem. 273(17):10107-10111.
59. Cronin C A, Gluba W, Scrable H., 2001, The lac operator-repressor system is functional in the mouse. Genes Dev. 15(12):1506-1517.
60. Hu M C, Davidson N, 1987, The inducible lac operator-repressor system is functional in mammalian cells. Cell. 48(4):555-566.
61. Figge J, Wright C, Collins C J, Roberts T M, Livingston D M., 1988, Stringent regulation of stably integrated chloramphenicol acetyl transferase genes by E. coli lac repressor in monkey cells. Cell. 52(5):713-722.

What is claimed is:

1. A recombinant vaccinia virus comprising:
a first recombinant nucleic acid comprising a first expression control sequence and a nucleic acid encoding a conditional replication gene product wherein the first expression control sequence is operably linked to the nucleic acid encoding the conditional replication gene product; and
a second recombinant nucleic acid comprising a second expression control sequence and a nucleic acid encoding a transcription factor that conditionally binds the first expression control sequence wherein the second expression control sequence is operably linked to the nucleic acid encoding a transcription factor,
and wherein the second recombinant nucleic acid is in a non-essential region of the vaccinia virus genome.

2. The recombinant vaccinia virus of claim 1, wherein the first expression control sequence comprises a tet response element and the transcription factor is selected from the group consisting of a tet repressor and a reverse tet repressor.

3. The recombinant vaccinia virus of claim 1, wherein the transcription factor is a tet repressor.

4. The recombinant vaccinia virus of claim 1, wherein the transcription factor is a reverse tet repressor.

5. The recombinant vaccinia virus of claim 1, wherein the first expression control sequence comprises a lac operator and the transcription factor is a lac repressor.

6. The recombinant vaccinia virus of claim 1, wherein the non-essential region of the vaccinia virus genome is the E2L/E3L inter-genic locus.

7. The recombinant vaccinia virus of claim 1, wherein the non-essential region of the vaccinia virus genome is the K1L/K2L inter-genic locus.

8. The recombinant vaccinia virus of claim 1, wherein the non-essential region of the vaccinia virus genome is the superoxide dismutase locus.

9. The recombinant vaccinia virus of claim 1, wherein the non-essential region of the vaccinia virus genome is the 7.5K locus.

10. The recombinant vaccinia virus of claim 1, wherein the first expression control sequence is a viral early/late promoter and the nucleic acid encoding a conditional replication gene product is an A14 gene.

11. The recombinant vaccinia virus of claim 1, wherein the first expression control sequence is a viral late promoter and the nucleic acid encoding a conditional replication gene product is a suicide gene selected from the group consisting of a constitutively-active cellular anti-viral human protein kinase p68 gene, an RNase A, a DNase I, an interferon-inducible nitric oxide synthase (iNOS), an eIF2α (S51D), an anti-sense A14R gene, a constitutively active caspase 3, and interferon-γ.

12. The recombinant vaccinia virus of claim 11, wherein the first recombinant nucleic acid is independently in (a) the E2L/E3L inter-genic locus, (b) the K1L/K2L inter-genic locus, (c) the superoxide dismutase locus, or (d) the 7.5K locus.

13. The recombinant vaccinia virus of claim 11, wherein the first recombinant nucleic acid is in the E2L/E3L inter-genic locus.

14. The recombinant vaccinia virus of claim 11, wherein the first recombinant nucleic acid is in the K1L/K2L inter-genic locus.

15. The recombinant vaccinia virus of claim 11, wherein the first recombinant nucleic acid is in the superoxide dismutase locus.

16. The recombinant vaccinia virus of claim 11, wherein the first recombinant nucleic acid is in the 7.5K locus.

17. A recombinant vaccinia virus comprising:
a nucleic acid comprising an expression control sequence and an exogenous nucleic acid encoding a conditional replication gene product,
wherein the expression control sequence is operably linked to the exogenous nucleic acid and wherein the nucleic acid is in a non-essential region of the vaccinia virus genome.

18. The recombinant vaccinia virus of claim 17, wherein the expression control sequence is a constitutive promoter and the exogenous nucleic acid is selected from the group consisting of a UL97 gene and an acyclovir-sensitivity gene.

19. The recombinant vaccinia virus of claim 18, wherein the non-essential region of the vaccinia virus genome is the E2L/E3L inter-genic locus.

20. The recombinant vaccinia virus of claim 18, wherein the non-essential region of the vaccinia virus genome is the K1L/K2L inter-genic locus.

21. The recombinant vaccinia virus of claim 18, wherein the non-essential region of the vaccinia virus genome is the superoxide dismutase locus.

22. The recombinant vaccinia virus of claim 18, wherein the non-essential region of the vaccinia virus genome is the 7.5K locus.

23. The recombinant vaccinia virus of claim 18, wherein the exogenous nucleic acid is a UL97 gene.

24. The recombinant vaccinia virus of claim 1S, wherein the exogenous nucleic acid is an acyclovir-sensitivity gene.

25. A vaccine against smallpox or vaccinia virus comprising a recombinant vaccinia virus according to claim 1 or claim 18.

26. A recombinant vaccinia virus comprising:
a first nucleic acid comprising a vaccinia virus early/late promoter and a tetR gene, wherein the vaccinia virus early/late promoter is operably linked to the tetR gene; and
a second nucleic acid comprising a recombinant A14 gene and a tet response element, wherein the tet response element is operably positioned between the A14 transcriptional start site and the A14 translational start site,
wherein the first nucleic acid is in a non-essential region of the vaccinia virus genome.

27. A method of vaccinating an individual against smallpox and vaccinia virus comprising:
administering to an individual the recombinant vaccinia virus of claim 26 in an amount sufficient to elicit an immune response; and
administering a derepressing amount of a drug selected from the group consisting of tetracycline, doxycycline, and minocycline.

28. The method of claim 27, wherein the individual is immunosuppressed.

29. A recombinant vaccinia virus comprising:
a first nucleic acid comprising a vaccinia virus early/late promoter and a reverse tetR gene, wherein the vaccinia virus early/late promoter is operably linked to the reverse tetR gene; and
a second nucleic acid comprising a recombinant A14 gene and a tet response element, wherein the tet response element is operably positioned between the A14 transcriptional start site and the A14 translational start site, wherein the first nucleic acid is in a non-essential region of the vaccinia virus genome.

30. A method of vaccinating an individual against smallpox and vaccinia virus comprising:
administering to an individual the recombinant vaccinia virus of claim 29 in an amount sufficient to elicit an immune response; and
optionally administering a repressing amount of a drug selected from the group consisting of tetracycline, doxycycline, and minocycline.

31. The method of claim 30, wherein the drug is administered to subjects displaying or at risk of displaying symptoms of viremia.

32. The method of claim 30, wherein the individual is immunosuppressed.

33. A recombinant vaccinia virus comprising:
a first nucleic acid comprising a vaccinia virus late promoter and a tetR gene, wherein the vaccinia virus late promoter is operably linked to the tetR gene; and
a second nucleic acid comprising an expression control sequence comprising a tet response element and the PKR gene, wherein the tet response element is operably linked to the PKR gene,
wherein the first and second nucleic acids are in non-essential regions of the vaccinia virus genome.

34. A method of vaccinating an individual against smallpox and vaccinia virus comprising:
administering to an individual the recombinant vaccinia virus of claim 33 in an amount sufficient to elicit an immune response; and
optionally administering a repressing amount of a drug selected from the group consisting of tetracycline, doxycycline, and minocycline.

35. The method of claim 34, wherein the drug is administered to subjects displaying or at risk of displaying symptoms of viremia.

36. The method of claim 34, wherein the individual is immunosuppressed.

37. A recombinant vaccinia virus comprising:
a first nucleic acid comprising a vaccinia virus early/late promoter and a reverse tetR gene, wherein the vaccinia virus early/late promoter is operably linked to the reverse tetR gene; and
a second nucleic acid comprising an expression control sequence comprising a tet response element and the PI<R gene, wherein the tet response element is operably linked to the PKR gene,
wherein the first and second nucleic acids are in non-essential regions of the vaccinia virus genome.

38. A method of vaccinating an individual against smallpox and vaccinia virus comprising:
administering to an individual the recombinant vaccinia virus of claim 37 in an amount sufficient to elicit an immune response; and
administering a derepressing amount of a drug selected from the group consisting of tetracycline, doxycycline, and minocycline.

39. The method of claim 38, wherein the individual is immunosuppressed.

40. A recombinant vaccinia virus comprising:
a nucleic acid comprising a vaccinia virus early/late promoter and a UL97 gene, wherein the vaccinia virus early/late promoter is operably linked to the UL97 gene,
wherein the nucleic acid is in a non-essential region of the vaccinia virus genome.

41. A recombinant vaccinia virus comprising:
a nucleic acid comprising a vaccinia virus early/late promoter and an acyclovir-sensitivity gene, wherein the vaccinia virus early/late promoter is operably linked to the acyclovir-sensitivity gene,
wherein the nucleic acid is in a non-essential region of the vaccinia virus genome.
